(19) United States
(12) Patent Application Publication
(10) Pub. No.: US 2025/0230401 A1
(43) Pub. Date: Jul. 17, 2025

(54) CELL MEMBRANE-BASED BIOLOGICAL MATERIAL FOR CROSS-SPECIES CELL COMPONENT DELIVERY AND PREPARATION METHOD THEREOF

(71) Applicant: ZHEJIANG UNIVERSITY, Hangzhou (CN)

(72) Inventors: Xianfeng LIN, Hangzhou (CN); Chenhui GU, Hangzhou (CN); Pengfei CHEN, Hangzhou (CN)

(21) Appl. No.: 19/060,733

(22) Filed: Feb. 23, 2025

Related U.S. Application Data
(63) Continuation of application No. PCT/CN2022/139154, filed on Dec. 14, 2022.

(30) Foreign Application Priority Data
Aug. 22, 2022 (CN) 202211004719.3

(51) Int. Cl. C12N 5/00; C12N 5/04; C12N 5/077

(52) U.S. Cl. CPC C12N 5/0012; C12N 5/04; C12N 5/0655

(57) ABSTRACT

Disclosed are a cell membrane-based biological material for cross-species cell component delivery and a preparation method thereof. The biological material is based on cell membrane coating, and a cross-species cell component is delivered into a specific cell in a membrane fusion mode, so that the cross-species component stably plays a specific function, thus providing a new thought and strategy for intracellular delivery of cross-species immunogenic biological materials.

### CROSS-REFERENCE TO RELATED APPLICATIONS

This application is a continuation of International Patent Application No. PCT/CN2022/139154 with a filing date of Dec. 14, 2022, designating the United States, now pending, and further claims priority to Chinese Patent Application No. 202211004719.3 with a filing date of Aug. 22, 2022. The content of the aforementioned applications, including any intervening amendments thereto, are incorporated herein by reference.

### TECHNICAL FIELD

The present invention relates to a biological material, and particularly to a cell membrane-based biological material for cross-species cell component delivery and a preparation method thereof.

### BACKGROUND OF THE PRESENT INVENTION

Effective intracellular delivery plays an important role in giving full play to the role of drugs and biological materials, and in case of involving immunogenic substances (especially cross-species components), shielding the immunogenicity is an indispensable link. Firstly, in organisms, various types of immune-related cells (mainly macrophages) are responsible for removing foreign matters. Secondly, at a subcellular level, lysosomes remove the foreign matters through degradation and digestion. Therefore, in the process of cross-species biological component delivery, an effective immune shielding method is needed.

As a coating carrier of cell system and a key cell structure for mutual recognition and regulation among various cells in vivo, a cell membrane contains immune immunity-related proteins such as a self-recognition receptor, and the cell membrane plays a vital role in preventing its internal contents from being eliminated, and may become a natural intracellular delivery material for immunogenic substances. In recent years, it has been reported that cell membrane-derived vesicles may be used to encapsulate specific materials, so as to enhance the biocompatibility and therapeutic effect.

At present, the application of cell membrane is mainly limited to the delivery of drugs, proteins, genes, and the like. The patent with the application number CN201810588367.8 discloses a preparation method of an anti-tumor therapeutic agent loaded by an autophagy-imitated immune cell, wherein the anti-tumor therapeutic agent is encapsulated with a cell membrane having an apoptosis group, so that the phagocytosis of the immune cell to the anti-tumor therapeutic agent is increased, but the method is limited to the delivery of drug nanoparticles for the treatment of tumor cell. The patent with the application number CN201810814853.7 discloses a preparation method of a prussian blue nanoparticle coated with a Ce6-embedded erythrocyte membrane, wherein the prussian blue nanoparticle is coated with photosensitizer Ce6-embedded erythrocyte membrane vesicle, so that the circulation time and biocompatibility of drugs in vivo are increased, but the method is only applicable to drug loading and does not focus on intracellular delivery. Such patents are based on the deimmunization of cell membrane and drug loading, without the design and preparation for the possibility of loading a cross-species cell component. Therefore, a cell membrane-based biological material for cross-species cell component delivery and a preparation method thereof are needed, and specific cell fusion is carried out through the cell membrane, so as to realize the intracellular delivery of the cross-species component, and enable the cross-species component to play the function in a specific cell.

### SUMMARY OF THE PRESENT INVENTION

Aiming at the defects in the prior art, the present invention provides a cell membrane-based biological material for cross-species cell component delivery and a preparation method thereof. The biological material is based on cell membrane coating, and a cross-species cell component is delivered into a specific cell in a membrane fusion mode, so that the cross-species component stably plays a specific function.

In order to achieve the objective above, the present invention provides the following technical solutions.

A cell membrane-based biological material for cross-species cell component delivery is provided, wherein the biological material is composed of a cell membrane of a target cell for delivery and a cross-species cell component encapsulated inside.

Preferably, the target cell for delivery comprises a motor system-related cell, a circulatory system-related cell, a digestive system-related cell, a urinary system-related cell, a nervous system-related cell, a germ cell, an endocrine cell and a tumor cell; the motor system-related cell comprises a chondrocyte, a myocyte, an osteoblast, an osteoclast and a mesenchymal stem cell; the circulatory system-related cell comprises a hematopoietic stem cell, a monocyte, a granulocyte, a macrophage, a B lymphocyte, a T lymphocyte, an erythrocyte, a platelet, a myocardial cell and a vascular endothelial cell; the digestive system-related cell comprises a hepatocyte, a gastrointestinal epithelial cell, a goblet cell and an islet cell; the urinary system-related cell comprises a respiratory system cell such as an alveolar cell and a tracheal epithelial cell, a glomerular endothelial cell and a renal tubular epithelial cell, and the nervous system-related cell comprises a neuronal cell, an astrocyte, an oligodendrocyte and a microglial cell.

Preferably, the target cell for delivery is a cell having definite differentiation characteristics, comprising a chondrocyte, a myocyte, a monocyte, a macrophage, an endothelial cell and an epithelial cell.

Preferably, the target cell for delivery is a chondrocyte having definite differentiation characteristics and targeting by intra-articular injection to avoid systemic influence.

Preferably, the cross-species cell refers to a cell having a different species source from the target cell, comprising an animal cell, a plant-derived cell, a bacterium and a fungus.

Preferably, the cross-species cell is a plant-derived cell.

Preferably, the cell component comprises a chloroplast and a content thereof, a nucleus and a content thereof, an endoplasmic reticulum and a content thereof, a Golgi apparatus and a content thereof, a mitochondrion and a content thereof, a ribosome and other cytoplasmic component.

Preferably, the cell component is thylakoid vesicle in chloroplast.

A preparation method of the cell membrane-based biological material for cross-species cell component delivery is provided, which specifically comprises the following steps of:
extracting the cross-species cell component, extracting the cell membrane of the target cell, and encapsulating the cross-species cell component into the cell membrane of the target cell, so as to obtain cell membrane vesicle encapsulating the cross-species cell component.

Preferably, the cross-species cell component is thylakoid vesicle, which is extracted in the following mode:
a plant green leaf material and a buffer A are mixed by a blender in a ratio of 1:1 to 1:10 (w/v); the obtained solution is filtered, and a filtrate is centrifuged at 3000 g for 10 minutes; a precipitate is gently resuspended in a buffer B; the solution is added to 80/40% Percoll gradient solution; a part containing a green layer is collected to obtain a thylakoid; a mass-volume ratio of the plant green leaf material to the buffer A is 1:1; ingredients of the buffer A are sorbitol, HEPES-KOH with pH of 7.6, MgCl₂ and 0.1% BSA; and ingredients of the buffer B are sorbitol, HEPES-KOH with pH of 7.6, MgCl₂, EDTA and L-sodium ascorbate, and a temperature of the buffer A is 4° C. to 10° C.

Preferably, the solution is filtered by pressing the solution to pass through a piece of fine mesh cotton fabric.

Preferably, a molar ratio of sorbitol, HEPES-KOH and MgCl₂ in the buffer A is 66:10:1.

Preferably, a molar ratio of sorbitol, HEPES-KOH with pH of 7.6, MgCl₂, EDTA and L-sodium ascorbate in the buffer B is 300:50:5:2: 10.

Preferably, a preparation method of the 80/40% Percoll gradient solution above is: 80% Percoll: 80% v/v Percoll, 10 mM L-sodium ascorbate, 300 mM sucrose and 66 mM MOPS-KOH with pH of 7.6; and 40% Percoll: 40% v/v Percoll, 10 mM L-sodium ascorbate, 300 mM sucrose and 25 mM MOPS-KOH with pH of 7.6.

Preferably, the preparation method of the cell membrane-based biological material for cross-species cell component delivery further comprises nanocrystallized granulation being ultrasonic extrusion nanocrystallization, which is specifically carried out in the following mode:
a thylakoid is subjected to an ultrasonic treatment in a bath ultrasonic instrument, and repeatedly extruded by a polycarbonate porous membrane; then the solution is centrifuged at 3000 g for 10 minutes; and a precipitate is resuspended in a buffer D, wherein ingredients of the buffer D are HEPES-KOH, MgCl₂ and sodium ascorbate.

Preferably, conditions of the ultrasonic treatment comprise: a No. 2 amplitude-change pole, 20% to 60% power, turning on for 2 seconds, turning off for 3 seconds, and working for 2 minutes.

Preferably, a pore size of the polycarbonate membrane above is 50 nm to 200 nm.

Preferably, the extraction of the cell membrane of the target cell is carried out in the following mode:
the cell is collected and resuspended in a buffer E at 4° C., and repeatedly whipped with an insulin needle for 20 times to lyse the cell, a cell homogenate and a 1 M sucrose buffer E solution are mixed in a ratio of 3:1 into a 0.25 M sucrose buffer E solution, the solution is centrifuged at 2000 g for 10 minutes, a supernatant is taken and centrifuged at 3000 g for 30 minutes, and a precipitate is the cell membrane; ingredients of the buffer E are mannitol, sucrose, Tris, MgCl₂, KCl, PMSF, a protease inhibitor without EDTA, DNase and RNase, and a pH value of the buffer E is 7.4; and a concentration of the Tris is 5 mM to 30 mM, and a concentration of the MgCl₂ is 1 mM to 20 mM.

Preferably, the concentration of the Tris is 10 mM.

Preferably, the concentration of the MgCl₂ is 1 mM.

Preferably, an encapsulating process is carried out in the following mode:
the cross-species cell component is loaded into the cell membrane vesicle by methods comprising microporous extrusion, ultrasonic hydration and a microfluidic technology.

Preferably, a filter-membrane microporous extrusion method is adopted; and a pore size of a filter membrane is 200 nm.

The present invention has the beneficial effects as follows:
1) According to the cell membrane-based biological material for cross-species cell component delivery provided by the present invention, the extracted cell membrane has a high reduction degree, and retains the fusion ability with the target cell.
2) According to the cell membrane-based biological material for cross-species cell component delivery provided by the present invention, there is an ideal double-layer vesicle configuration, which can realize the cross-species cell component delivery based on membrane fusion.
3) According to the cell membrane-based biological material for cross-species cell component delivery provided by the present invention, there is high selectivity for the target cell, leading to a low off-target rate.
4) According to the cell membrane-based biological material for cross-species cell component delivery provided by the present invention, lysosome escape may be achieved by membrane fusion delivery, so that the cross-species cell component stably exists in target cytoplasm.

### DESCRIPTION OF THE DRAWINGS

In order to make the objectives, technical solutions and beneficial effects of the present invention clearer, the present invention provides the following drawings for description:

FIG. 1 shows western blotting of a chondrocyte membrane in Embodiment 1, indicating that the extracted cell membrane retains a membrane-associated protein and removes a cytoplasmic protein.

FIG. 2 shows protein mass spectrometry analysis of the chondrocyte membrane in Embodiment 1, indicating that components of the extracted cell membrane conform to theoretical composition.

FIG. 3 shows mass spectrometry analysis and quantification of proteins related to vesicle targeting and membrane fusion of the chondrocyte membrane in Embodiment 1, indicating that the extracted cell membrane retains a fusion ability with an original cell.

FIG. 4(a) and FIG. 4(b) show cryo-electron micrographs of a cell membrane vesicle and a cell membrane-encapsulated nano-thylakoid vesicle of Embodiment 1, indicating that an ideal double-layer vesicle is presented after encapsulation.

FIG. 5(a) and FIG. 5(b) show flow cytometry analysis of uptake degrees of chondrocyte to a chondrocyte membrane-encapsulated nano-thylakoid in Embodiment 1 under the conditions of endocytosis inhibitor and low-temperature inhibition, i indicating that a delivery mode of the chondrocyte membrane-encapsulated nano-thylakoid is membrane fusion delivery instead of endocytosis uptake.

FIG. 6(a) to FIG. 6(d) show comparison of fluoroscopic images of a chondrocyte lysosome, a liposome-encapsulated nano-thylakoid ingested into a chondrocyte and a chondrocyte membrane-encapsulated nano-thylakoid in Embodiment 1, indicating that the liposome-encapsulated nano-thylakoid enters the lysosome, while the chondrocyte membrane-encapsulated nano-thylakoid realizes lysosome escape and stably exists in cytoplasm.

### DETAILED DESCRIPTION OF PREFERRED EMBODIMENTS

A cell membrane-based biological material for cross-species cell component delivery and a preparation method thereof provided by the present invention are described in detail hereinafter with reference to embodiments, but the embodiments cannot be understood as limiting the scope of protection of the present invention.

According to the cell membrane-based biological material for cross-species cell component delivery provided by the present invention, the biological material is composed of a cell membrane of a specific cell and a cross-species cell component encapsulated inside. The “specific cell” refers to a target cell for delivery, comprising a motor system-related cell such as a chondrocyte, a myocyte, an osteoblast, an osteoclast and a mesenchymal stem cell; a circulatory system-related cell such as a hematopoietic stem cell, a monocyte, a granulocyte, a macrophage, a B lymphocyte, a T lymphocyte, an erythrocyte, a platelet, a myocardial cell and a vascular endothelial cell, a digestive system-related cell such as a hepatocyte, a gastrointestinal epithelial cell, a goblet cell and an islet cell, a urinary system-related cell such as, a respiratory system cell such as an alveolar cell and a tracheal epithelial cell, a glomerular endothelial cell and a renal tubular epithelial cell, a nervous system-related cell such as a neuronal cell, an astrocyte, an oligodendrocyte and a microglial cell, and a germ cell, an endocrine cell, a tumor cell, and the like. Preferably, the cell is a cell having definite differentiation characteristics, comprising a chondrocyte, a myocyte, a monocyte, a macrophage, an endothelial cell and an epithelial cell, and more preferably, the cell is a chondrocyte having definite differentiation characteristics and targeting by intra-articular injection to avoid systemic influence.

The “cell membrane” refers to a cell membrane of the target cell, comprising a cell membrane of the cell in the above term “specific cell”. Preferably, the cell membrane is a cell membrane of the cell having definite differentiation characteristics, comprising the chondrocyte, the myocyte, the monocyte, the macrophage, the endothelial cell and the epithelial cell, and more preferably, the cell membrane is a cell membrane of the chondrocyte having definite differentiation characteristics and targeting by intra-articular injection to avoid systemic influence. The “cross-species cell” refers to a cell having a different species source from the target cell, comprising an animal cell, such as a cell of human, pig, cow, sheep or mouse, a plant-derived cell, a bacterium and a fungus. Preferably, the cross-species cell is the plant-derived cell. The “cell component” comprises a chloroplast and a content thereof, a nucleus and a content thereof, an endoplasmic reticulum and a content thereof, a Golgi apparatus and a content thereof, a mitochondrion and a content thereof, a ribosome and other cytoplasmic component. Preferably, the cell component is thylakoid vesicle in chloroplast.

The preparation method of the cell membrane-based biological material for cross-species cell component delivery provided by the present invention comprises the following steps.

In step 1, the cross-species cell component, preferably a plant thylakoid, is extracted in the following mode.

A plant green leaf material and a cold buffer A are mixed by a blender in a ratio of 1:1 to 1:10 (w/v). The obtained solution is pressed to pass through a piece of fine mesh cotton fabric, and a filtrate is centrifuged at 3000 g for 10 minutes. A precipitate is gently resuspended in a buffer B. The solution is added to 80/40% Percoll gradient solution. A part containing a green layer is collected to obtain a thylakoid. Ingredients of the buffer A above are 330 mM sorbitol, 50 mM HEPES-KOH with pH of 7.6, 5 mM MgCl₂ and 0.1% BSA. Ingredients of the buffer B above are 300 mM sorbitol, 50 mM HEPES-KOH with pH of 7.6, 5 mM MgCl₂, 2 mM EDTA and 10 mM L-sodium ascorbate. A preparation method of the 80/40% Percoll gradient solution above is: 80% Percoll: 80% v/v Percoll, 10 mM L-sodium ascorbate, 300 mM sucrose and 66 mM MOPS-KOH with pH of 7.6; and 40% Percoll: 40% v/v Percoll, 10 mM L-sodium ascorbate, 300 mM sucrose and 25 mM MOPS-KOH with pH of 7.6.

In step 2, a granulating process, preferably an ultrasonic extrusion nanocrystallization process, is carried out in the following mode.

The thylakoid is subjected to an ultrasonic treatment in a bath ultrasonic instrument, and repeatedly extruded by a polycarbonate porous membrane. Then, the solution is centrifuged at 3000 g for 10 minutes. A precipitate is resuspended in a buffer D. Conditions of the ultrasonic treatment above comprise: a No. 2 amplitude-change pole, 20% to 60% power, turning on for 2 seconds, turning off for 3 seconds, and working for 2 minutes, and preferably 40% power. A pore size of the polycarbonate membrane above is 50 nm to 200 nm, and preferably, the pore size is 100 nm. Ingredients of the buffer D above are 10 mM HEPES-KOH, 10 mM MgCl₂ and 10 mM L-sodium ascorbate.

In step 3, the extraction of the specific cell membrane is carried out in the following mode.

The cell is collected and resuspended in a buffer E at 4° C., and repeatedly whipped with an insulin needle for 20 times to lyse the cell, a cell homogenate and a 1 M sucrose buffer E solution are mixed in a ratio of 3:1 into a 0.25 M sucrose buffer E solution, the solution is centrifuged at 2000 g for 10 minutes, a supernatant is taken and centrifuged at 3000 g for 30 minutes, and a precipitate is the cell membrane. Ingredients of the buffer E above are mannitol, sucrose, Tris, MgCl₂, KCl, PMSF, a protease inhibitor without EDTA, DNase and RNase, and a pH value of the buffer E is 7.4. Preferably, a concentration of the Tris is 5 mM to 30 mM, and more preferably, the concentration of the Tris is 10 mM. Preferably, a concentration of the MgCl₂ is 1 mM to 20 mM, and more preferably the concentration of the MgCl₂ is 1 mM.

In step 4, an encapsulating process is carried out in the following mode.

The cross-species cell component is loaded into the cell membrane vesicle by methods comprising microporous extrusion, ultrasonic hydration and a microfluidic technology. Preferably, a filter-membrane microporous extrusion method is adopted. More preferably, a pore size of a filter membrane is 200 nm.

The present invention may also adopt other cross-species cell components, other specific cell preparation schemes, other specific cell membranes, and other encapsulating processes mentioned above, all of which can achieve the same technical effects.

### Embodiment 1. Encapsulation of Plant Thylakoid with Chondrocyte Membrane by Filter-Membrane Microporous Extrusion Method

In step 1, a cross-species cell component of a plant thylakoid was extracted in the following mode.

1 g of plant green leaf material and 1 mL of cold buffer A were mixed by a blender. The obtained solution was pressed to pass through a piece of fine mesh cotton fabric, and a filtrate was centrifuged at 3000 g for 10 minutes. A precipitate was gently resuspended in a buffer B. The solution was added to 80/40% Percoll gradient solution. A part containing a green layer was collected to obtain a thylakoid. Ingredients of the buffer A above were 330 mM sorbitol, 50 mM HEPES-KOH with pH of 7.6, 5 mM MgCl₂ and 0.1% BSA. Ingredients of the buffer B above were 300 mM sorbitol, 50 mM HEPES-KOH with pH of 7. 6, 5 mM MgCl₂, 2 mM EDTA and 10 mM L-sodium ascorbate. A preparation method of the 80/40% Percoll gradient solution above was: 80% Percoll: 80% v/v Percoll, 10 mM L-sodium ascorbate, 300 mM sucrose and 66 mM MOPS-KOH with pH of 7.6; and 40% Percoll: 40% v/v Percoll, 10 mM L-sodium ascorbate, 300 mM sucrose and 25 mM MOPS-KOH with pH of 7.6.

In step 2, an ultrasonic extrusion nanocrystallization process was carried out in the following mode.

The thylakoid was subjected to an ultrasonic treatment in a bath ultrasonic instrument, and repeatedly extruded by a 100 nm polycarbonate porous membrane. Then, the solution was centrifuged at 3000 g for 10 minutes. A precipitate was resuspended in a buffer D. Conditions of the ultrasonic treatment above comprised: a No. 2 amplitude-change pole, 40% power, turning on for 2 seconds, turning off for 3 seconds, and working for 2 minutes. Ingredients of the buffer D above were 10 mM HEPES-KOH, 10 mM MgCl₂ and 10 mM L-sodium ascorbate.

In step 3, the extraction of the chondrocyte membrane was carried out in the following mode.

The chondrocyte was collected and resuspended in a buffer E at 4° C., and repeatedly whipped with an insulin needle for 20 times to lyse the cell, a cell homogenate and a 1 M sucrose buffer E solution were mixed in a ratio of 3:1 into a 0.25 M sucrose buffer E solution, the solution was centrifuged at 2000 g for 10 minutes, a supernatant was taken and centrifuged at 3000 g for 30 minutes, and a precipitate was the cell membrane. Ingredients of the buffer E above were mannitol, sucrose, Tris, MgCl₂, KCl, PMSF, a protease inhibitor without EDTA, DNase and RNase, and a pH value of the buffer E was 7.4. A concentration of the Tris was 10 mM, and a concentration of the MgCl₂ was 1 mM.

In step 4, an encapsulating process was carried out in the following mode.

The thylakoid was blended with the chondrocyte membrane, and repeatedly extruded through a 200 nm polycarbonate membrane to prepare the chondrocyte membrane-encapsulated plant thylakoid.

### Embodiment 2. Encapsulation of Plant Thylakoid with Hepatocyte Membrane by Ultrasonic Hydration Method

In step 1, a cross-species cell component of a plant thylakoid was extracted in the following mode.

1 g of plant green leaf material and 2 mL of cold buffer A were mixed by a blender. The obtained solution was pressed to pass through a piece of fine mesh cotton fabric, and a filtrate was centrifuged at 3000 g for 10 minutes. A precipitate was gently resuspended in a buffer B. The solution was added to 80/40% Percoll gradient solution. A part containing a green layer was collected to obtain a thylakoid. Ingredients of the buffer A above were 330 mM sorbitol, 50 mM HEPES-KOH with pH of 7.6, 5 mM MgCl₂ and 0.1% BSA. Ingredients of the buffer B above were 300 mM sorbitol, 50 mM HEPES-KOH with pH of 7.6, 5 mM MgCl₂, 2 mM EDTA and 10 mM L-sodium ascorbate. A preparation method of the 80/40% Percoll gradient solution above was: 80% Percoll: 80% v/v Percoll, 10 mM L-sodium ascorbate, 300 mM sucrose and 66 mM MOPS-KOH with pH of 7.6; and 40% Percoll: 40% v/v Percoll, 10 mM L-sodium ascorbate, 300 mM sucrose and 25 mM MOPS-KOH with pH of 7.6.

In step 2, an ultrasonic extrusion nanocrystallization process was carried out in the following mode.

The thylakoids was subjected to an ultrasonic treatment in a bath ultrasonic instrument, and repeatedly extruded by a 100 nm polycarbonate porous membrane. Then, the solution was centrifuged at 3000 g for 10 minutes. A precipitate was resuspended in a buffer D. Conditions of the ultrasonic treatment above comprised: a No. 2 amplitude-change pole, 40% power, turning on for 2 seconds, turning off for 3 seconds, and working for 2 minutes. Ingredients of the buffer D above were 10 mM HEPES-KOH, 10 mM MgCl₂ and 10 mM L-sodium ascorbate.

In step 3, the extraction of the hepatocyte membrane was carried out in the following mode.

The hepatocyte was collected and resuspended in a buffer E at 4° C., and repeatedly whipped with an insulin needle for 20 times to lyse the cell, a cell homogenate and a 1 M sucrose buffer E solution were mixed in a ratio of 3:1 into a 0.25 M sucrose buffer E solution, the solution was centrifuged at 2000 g for 10 minutes, a supernatant was taken and centrifuged at 3000 g for 30 minutes, and a precipitate was the cell membrane. Ingredients of the buffer E above were mannitol, sucrose, Tris, MgCl₂, KCl, PMSF, a protease inhibitor without EDTA, DNase and RNase, and a pH value of the buffer E was 7.4. A concentration of the Tris was 10 mM, and a concentration of the MgCl₂ was 1 mM.

In step 4, an encapsulating process was carried out in the following mode.

The hepatocyte membrane was dissolved in ethanol, and spin-dried in a vacuum spin dryer, and the solution containing the plant thylakoid was added into a flask, and subjected to bath ultrasonic hydration, so as to prepare the hepatocyte membrane-encapsulated plant thylakoid.

### Embodiment 3. Encapsulation of Plant Cytoplasmic Protein with Monocyte Membrane by Microfluidic Method

In step 1, a cross-species cell component of a plant cytoplasmic protein was extracted in the following mode.

1 g of plant green leaf material and 5 mL of cold buffer A were mixed by a blender. The obtained solution was pressed to pass through a piece of fine mesh cotton fabric, and a filtrate was centrifuged at 3000 g for 10minutes. A supernatant was added with the same amount of ethanol and ¼ amount of chloroform, and mixed evenly. The mixture was centrifuged at 12000 rpm for 5 minutes, and an upper layer was discarded. The product was added with 500 ul of ethanol, mixed evenly in a translational manner, and centrifuged at 12000 rpm for 5 minutes, an upper layer was discarded, and a precipitate was resuspended to obtain the cytoplasmic protein. Ingredients of the buffer A above were 330 mM sorbitol, 50 mM HEPES-KOH with pH of 7.6, 5 mM MgCl₂ and 0.1% BSA.

In step 2, the extraction of the monocyte membrane was carried out in the following mode.

The monocyte was collected and resuspended in a buffer E at 4° C., and repeatedly whipped with an insulin needle for 20 times to lyse the cell, a cell homogenate and a 1 M sucrose buffer E solution were mixed in a ratio of 3:1 into a 0.25 M sucrose buffer E solution, the solution was centrifuged at 2000 g for 10 minutes, a supernatant was taken and centrifuged at 3000 g for 30 minutes, and a precipitate was the cell membrane. Ingredients of the buffer E above were mannitol, sucrose, Tris, MgCl₂, KCl, PMSF, a protease inhibitor without EDTA, DNase and RNase, and a pH value of the buffer E was 7.4. A concentration of the Tris was 10 mM, and a concentration of the MgCl₂ was 1 mM.

In step 3, an encapsulating process was carried out in the following mode.

The monocyte membrane was dissolved in ethanol, the plant cytoplasmic protein was dissolved in water, the plant cytoplasmic protein aqueous solution was encapsulated into a cell membrane-containing liquid flow through a microfluidic V-chip, and the ethanol was removed through dialysis, so as to prepare the monocyte membrane-encapsulated plant cytoplasmic protein.

### Western Blotting of Chondrocyte Membrane in Embodiment 1

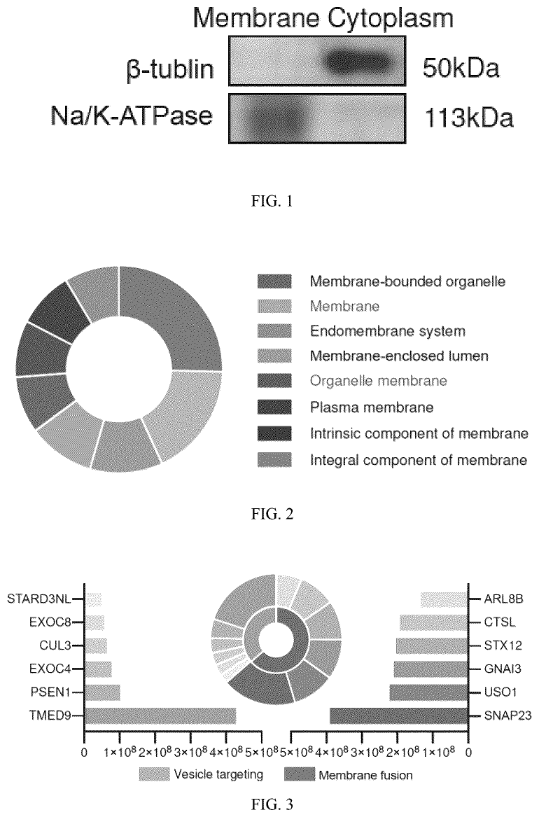

1. A Lysis Buffer was used to lyse the chondrocyte membrane, and a WB western blotting experiment was carried out.
2. Bands were cut according to molecular weights of corresponding proteins, and a primary antibody and a secondary antibody were incubated respectively.
3. A membrane protein Na/K-ATPase and a cytoplasmic protein β-actin could be seen after chemiluminescence exposure. It was indicated that the extracted cell membrane retained the membrane-related protein and removed the cytoplasmic protein, as shown in FIG. 1.

### Protein Mass Spectrometry Analysis of Chondrocyte Membrane in Embodiment 1

1. The extracted cell membrane was subjected to protein extraction, and then subjected to protein mass spectrometry analysis.
2. Results of the mass spectrometry showed that components of the extracted cell membrane conformed to theoretical composition, and mass spectrometry analysis and quantification of proteins related to vesicle targeting and membrane fusion of the chondrocyte membrane showed that the extracted cell membrane retained a fusion ability with an original cell, as shown in FIG. 2 to FIG. 3.

### Cryo-Electron Micrographs of Cell Membrane Vesicle and Cell Membrane-Encapsulated Nano-Thylakoid Vesicle in Embodiment 1

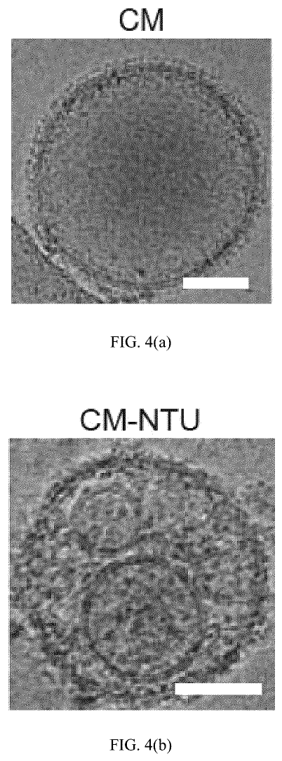

1. The cell membrane vesicle and the cell membrane-encapsulated nano-thylakoid vesicle were subjected to cryo-electron microscopic sample preparation respectively, and observed respectively.
2. The cryo-electron micrographs showed that ideal double-layer vesicle was presented after encapsulation, as shown in FIG. 4(a) and FIG. 4(b).

### Cytometry Analysis of Uptake Degrees of Chondrocyte to Chondrocyte Membrane-Encapsulated Nano-Thylakoid in Embodiment 1 Under Conditions of Endocytosis Inhibitor and Low-Temperature Inhibition

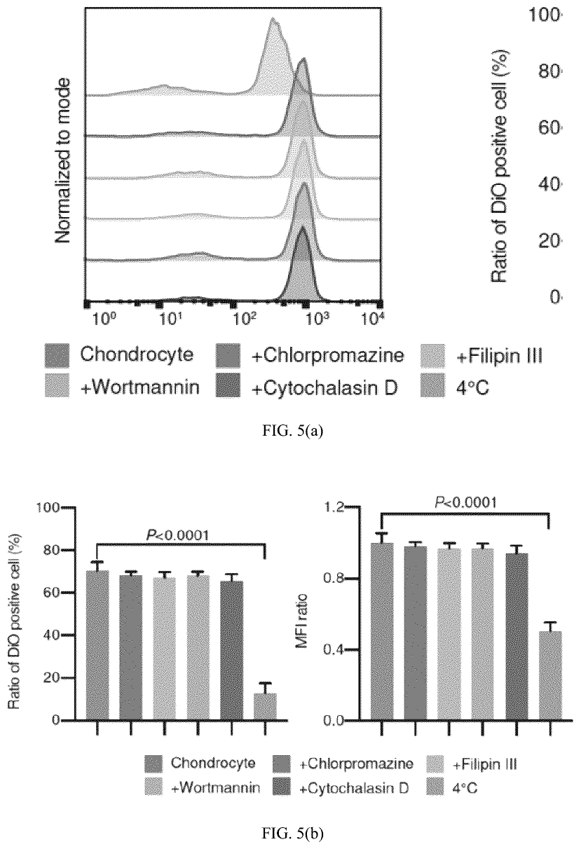

1. The chondrocyte membrane-encapsulated nano-thylakoid was added into a culture medium of chondrocyte cultured, and groups were set for adding the endocytosis inhibitor as shown in the figure and carrying out the low temperature treatment respectively.
2. The digestive cell was subjected to flow cytometry.
3. Results showed that the uptake was decreased only in the low-temperature treatment group, and the endocytosis inhibitor did not affect the uptake, indicating that the delivery mode of the chondrocyte membrane-encapsulated nano-thylakoid was membrane fusion delivery instead of endocytosis uptake, as shown in FIG. 5(a) and FIG. 5(b).

### Comparison of Fluoroscopic Images of Chondrocyte Lysosome, Liposome-Encapsulated Nano-Thylakoid Ingested into Chondrocyte and Chondrocyte Membrane-Encapsulated Nano-Thylakoid in Embodiment 1

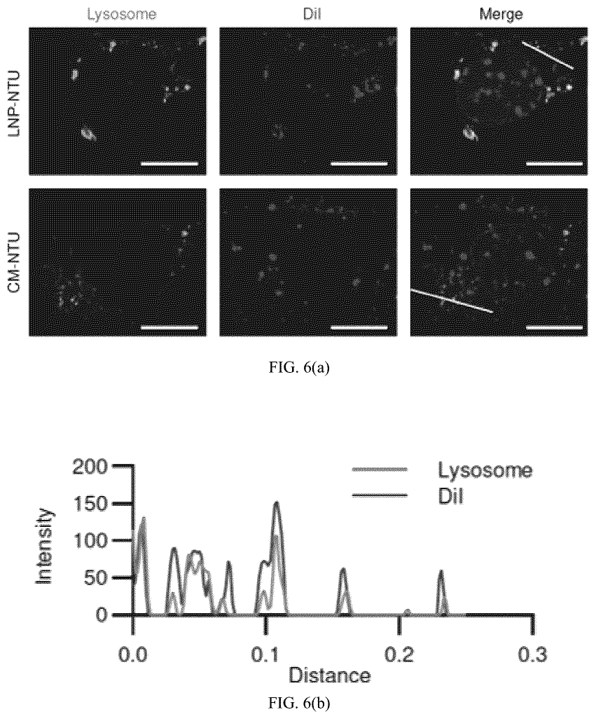

1. The liposome-encapsulated nano-thylakoid and the chondrocyte membrane-encapsulated nano-thylakoid were respectively added into a culture medium of chondrocyte cultured.
2. Laser confocal microscope imaging was carried out, indicating that the liposome-encapsulated nano-thylakoid entered the lysosome, while the chondrocyte membrane-encapsulated nano-thylakoid realized lysosome escape and stably existed in cytoplasm, as shown in FIG. 6(a) to FIG. 6(d).

The cell membrane-based biological materials for cross-species cell component delivery obtained in Embodiments 2 and 3 were subjected to the cell-membrane western blotting, the protein mass spectrometry analysis and quantification, the cryo-electron microscopy after encapsulation, the endocytosis inhibition experiment and the lysosome escape experiment respectively, and the results were similar to those obtained by the filter-membrane microporous extrusion method with the chondrocyte membrane in Embodiment 1, which indicated that the preparation of the cell membrane-based biological material for cross-species cell component delivery could be achieved by other cross-species cell components, other specific cell preparation schemes, other specific cell membranes, and other encapsulating processes mentioned above.

The above are only the preferred embodiments of the present invention, and it should be pointed out that, although the present invention has been described in detail with reference to the preferred embodiments above, those skilled in the art should understand that several improvements and decorations may also be made without departing from the principle of the present invention, and these improvements and decorations should also be regarded as falling within the scope of protection of the present invention without departing from the scope defined by the claims of the present invention.

We claim:

1. A cell membrane-based biological material for cross-species cell component delivery, wherein the biological material is composed of a cell membrane of a target cell for delivery and a cross-species cell component encapsulated inside.

2. A cell membrane-based biological material for cross-species cell component delivery, wherein a target cell for delivery comprises a motor system-related cell, a circulatory system-related cell, a digestive system-related cell, a urinary system-related cell, a nervous system-related cell, a germ cell, an endocrine cell and a tumor cell; the motor system-related cell comprises a chondrocyte, a myocyte, an osteoblast, an osteoclast and a mesenchymal stem cell; the circulatory system-related cell comprises a hematopoietic stem cell, a monocyte, a granulocyte, a macrophage, a B lymphocyte, a T lymphocyte, an erythrocyte, a platelet, a myocardial cell and a vascular endothelial cell; the digestive system-related cell comprises a hepatocyte, a gastrointestinal epithelial cell, a goblet cell and an islet cell; the urinary system-related cell comprises a respiratory system cell such as an alveolar cell and a tracheal epithelial cell, a glomerular endothelial cell and a renal tubular epithelial cell; and the nervous system-related cell comprises a neuronal cell, an astrocyte, an oligodendrocyte and a microglial cell.

3. The cell membrane-based biological material for cross-species cell component delivery according to claim 1, wherein the target cell for delivery is a cell having definite differentiation characteristics, comprising a chondrocyte, a myocyte, a monocyte, a macrophage, an endothelial cell and an epithelial cell.

4. The cell membrane-based biological material for cross-species cell component delivery according to claim 1, wherein the target cell for delivery is a chondrocyte having definite differentiation characteristics and targeting by intra-articular injection to avoid systemic influence.

5. The cell membrane-based biological material for cross-species cell component delivery according to claim 1, wherein the cross-species cell refers to a cell having a different species source from the target cell, comprising an animal cell, a plant-derived cell, a bacterium and a fungus.

6. The cell membrane-based biological material for cross-species cell component delivery according to claim 1, wherein the cross-species cell is a plant-derived cell.

7. The cell membrane-based biological material for cross-species cell component delivery according to claim 1, wherein the cell component comprises a chloroplast and a content thereof, a nucleus and a content thereof, an endoplasmic reticulum and a content thereof, a Golgi apparatus and a content thereof, a mitochondrion and a content thereof, a ribosome and other cytoplasmic component.

8. The cell membrane-based biological material for cross-species cell component delivery according to claim 1, wherein the cell component is thylakoid vesicle in chloroplast.

9. A preparation method of the cell membrane-based biological material for cross-species cell component delivery according to claim 1, specifically comprising the following steps of:
extracting the cross-species cell component, extracting the cell membrane of the target cell, and encapsulating the cross-species cell component into the cell membrane of the target cell, so as to obtain cell membrane vesicle encapsulating the cross-species cell component.

10. A preparation method of the cell membrane-based biological material for cross-species cell component delivery according to claim 1, wherein the cross-species cell component is thylakoid vesicle, which is extracted in the following mode:
a plant green leaf material and a buffer A are mixed by a blender in a ratio of 1:1 to 1:10 w/v; the obtained solution is filtered, and a filtrate is centrifuged at 3000 g for 10 minutes; a precipitate is gently resuspended in a buffer B; the solution is added to 80/40% Percoll gradient solution; a part containing a green layer is collected to obtain a thylakoid; a mass-volume ratio of the plant green leaf material to the buffer A is 1:1; ingredients of the buffer A are sorbitol, HEPES-KOH with pH of 7.6, MgCl₂ and 0.1% BSA; and ingredients of the buffer B are sorbitol, HEPES-KOH with pH of 7.6, MgCl₂, EDTA and L-sodium ascorbate, and a temperature of the buffer A is 4° C. to 10° C.

11. The preparation method of the cell membrane-based biological material for cross-species cell component delivery according to claim 9, further comprising nanocrystallized granulation being ultrasonic extrusion nanocrystallization, which is specifically carried out in the following mode:
a thylakoid is subjected to an ultrasonic treatment in a bath ultrasonic instrument, and repeatedly extruded by a polycarbonate porous membrane; then the solution is centrifuged at 3000 g for 10 minutes; and a precipitate is resuspended in a buffer D, wherein ingredients of the buffer D are HEPES-KOH, MgCl₂ and sodium ascorbate.

12. The preparation method of the cell membrane-based biological material for cross-species cell component delivery according to claim 11, wherein conditions of the ultrasonic treatment comprise: a No. 2 amplitude-change pole, 20% to 60% power, turning on for 2 seconds, turning off for 3 seconds, and working for 2 minutes.

13. The preparation method of the cell membrane-based biological material for cross-species cell component delivery according to claim 11, wherein a pore size of the polycarbonate membrane above is 50 nm to 200 nm.

14. The preparation method of the cell membrane-based biological material for cross-species cell component delivery according to claim 9, wherein the extraction of the cell membrane of the target cell is carried out in the following mode:
the cell is collected and resuspended in a buffer E at 4° C., and repeatedly whipped with an insulin needle for 20 times to lyse the cell, a cell homogenate and a 1 M sucrose buffer E solution are mixed in a ratio of 3:1 into a 0.25 M sucrose buffer E solution, the solution is centrifuged at 2000 g for 10 minutes, a supernatant is taken and centrifuged at 3000 g for 30 minutes, and a precipitate is the cell membrane; ingredients of the buffer E are mannitol, sucrose, Tris, MgCl₂, KCl, PMSF, a protease inhibitor without EDTA, DNase and RNase, and a pH value of the buffer E is 7.4; and a concentration of the Tris is 5 mM to 30 mM, and a concentration of the MgCl₂ is 1 mM to 20 mM.

15. The preparation method of the cell membrane-based biological material for cross-species cell component delivery according to claim 14, wherein the concentration of the Tris is 10 mM.

16. The preparation method of the cell membrane-based biological material for cross-species cell component delivery according to claim 14, wherein the concentration of the MgCl₂ is 1 mM.

17. The preparation method of the cell membrane-based biological material for cross-species cell component delivery according to claim 9, wherein an encapsulating process is carried out in the following mode:
the cross-species cell component is loaded into the cell membrane vesicle by methods comprising microporous extrusion, ultrasonic hydration and a microfluidic technology.

18. The preparation method of the cell membrane-based biological material for cross-species cell component delivery according to claim 17, wherein a filter-membrane microporous extrusion method is adopted; and a pore size of a filter membrane is 200 nm.
